(19) United States
(12) Patent Application Publication
(10) Pub. No.: US 2024/0151603 A1
(43) Pub. Date: May 9, 2024

(54) VAPOR PRESSURE DEFICIT SENSOR

(71) Applicant: HGCI, Inc., Las Vegas, NV (US)

(72) Inventors: Dengke Cai, Camas, WA (US); Wen Zhang, Ridgefield, WA (US); Yinlin Li, Camas, WA (US)

(21) Appl. No.: 18/505,938

(22) Filed: Nov. 9, 2023

Related U.S. Application Data
(60) Provisional application No. 63/424,106, filed on Nov. 9, 2022.

(51) Int. Cl. G01L 19/00; G01L 19/14

(52) U.S. Cl. CPC G01L 19/0038; G01L 19/0092; G01L 19/142

(57) ABSTRACT

A VPD sensor for a horticultural environment is provided. The VPD sensor includes an upper housing, a lower housing, and a vapor pressure deficit sensing module. The lower housing is coupled with the upper housing and includes a lower sidewall and a bottom wall that cooperate to define an interior. The vapor pressure deficit sensing module is coupled with the lower housing and is disposed in the interior. The upper housing and the lower housing are spaced from each other along a centerline to define an air gap therebetween that is in fluid communication with the interior. The lower sidewall defines an opening that is in fluid communication with the interior and cooperates with the interior and the air gap to define a fluid pathway that extends through the interior and between the opening and the air gap.

### REFERENCE TO RELATED APPLICATION

This application claims priority of U.S. provisional patent application Ser. No. 63/424,106 filed Nov. 9, 2022, and hereby incorporates this patent application by reference herein in its entirety.

### TECHNICAL FIELD

The apparatus described below generally relates to a VPD sensor for a horticultural environment. In particular, the VPD sensor can be configured to detect an environmental parameter of a soil substrate to facilitate notification to a user when the environmental parameter is out of range.

### BACKGROUND

When plants are grown in an indoor horticultural environment, such as a greenhouse, the vapor pressure deficit of the surrounding atmosphere can affect the viability and growth of the plants. A vapor pressure deficit sensor can be installed in the indoor horticultural environment that detects the vapor pressure deficit and provides an indicator to a user to allow the user to intervene when the vapor pressure deficit is outside of a desired range.

### BRIEF DESCRIPTION OF THE DRAWINGS

Various embodiments will become better understood with regard to the following description, appended claims and accompanying drawings wherein:

FIG. 1 is an upper front isometric view depicting a VPD sensor;

FIG. 2 is a lower front isometric view depicting the VPD sensor of FIG. 1;

FIG. 3 is an exploded view of the VPD sensor of FIG. 1;

FIG. 4 is a schematic view of the VPD sensor of FIG. 1 in association with a remote controller; and

FIG. 5 is a sectional view taken along the line 5-5 of FIG. 1.

### DETAILED DESCRIPTION

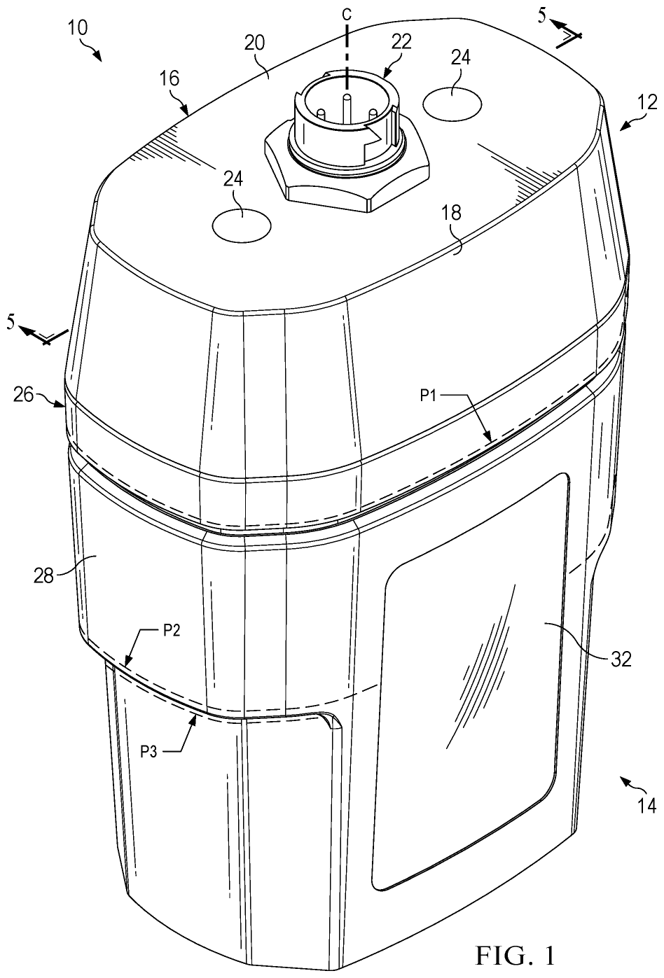

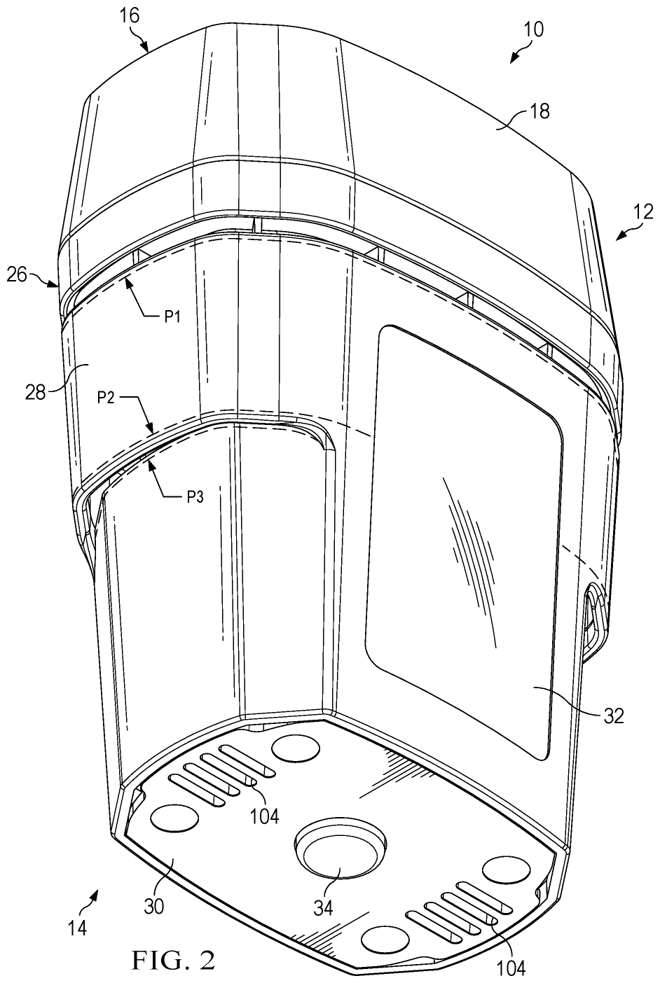

Embodiments are hereinafter described in detail in connection with the views and examples of FIGS. 1-5, wherein like numbers indicate the same or corresponding elements throughout the views. A vapor pressure deficit (VPD) sensor 10 is generally depicted in FIGS. 1 and 2 and is shown to include an upper housing 12 and a lower housing 14 that are coupled together. The upper housing 12 can include an outer shroud 16 that includes a sidewall 18 and a top wall 20 (FIG. 1). An electrical connector 22 and a pair of light emitting diodes (LEDs) 24 (FIG. 1) can be disposed at the top wall 20 and a light ring 26 can be disposed beneath the sidewall 18 and can extend circumferentially around the sidewall 18. The lower housing 14 can include a sidewall 28 and a bottom wall 30 (FIG. 2). The sidewall 28 can support a display 32 and the bottom wall 30 can support a pushbutton 34 (FIG. 2).

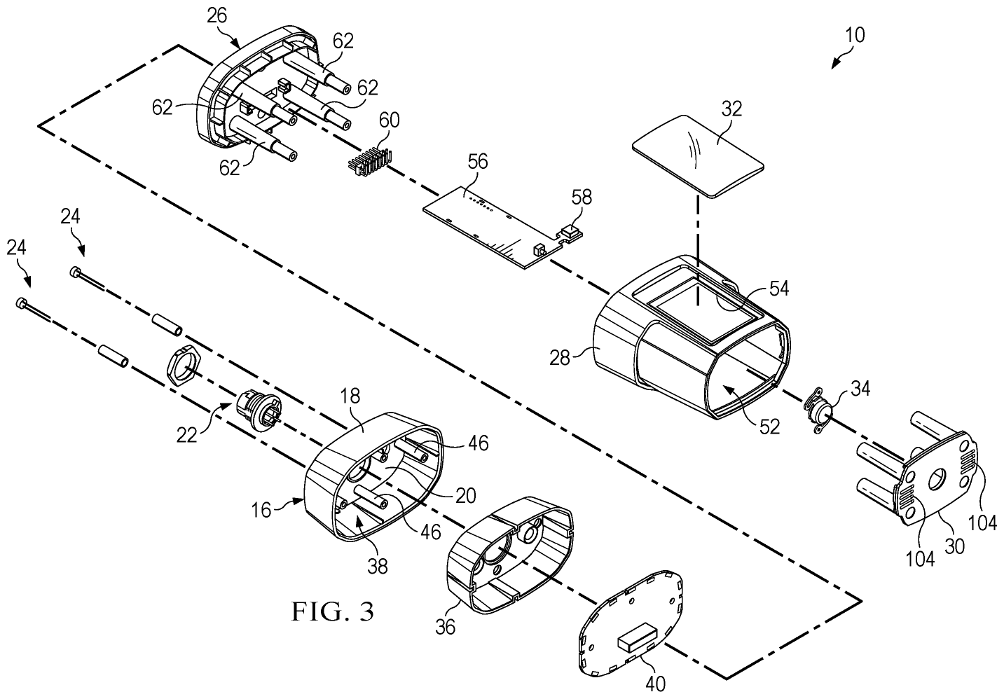

Referring now to FIG. 3, an inner shroud 36 can be disposed within an inner cavity 38 defined by the outer shroud 16 and can cooperate with the outer shroud 16 to support a printed circuit board (PCB) 40. The PCB 40 can electrically support the electrical connector 22 and the pair of LEDs 24. The PCB 40 can be sandwiched between the inner shroud 36 and the light ring 26. The outer shroud 16, the inner shroud 36, the PCB 40 and the light ring 26 can be secured together with screws (not shown) that extend through the light ring 26, the PCB 40, and the inner shroud 36 and are threaded into standoffs 46 that extend from the outer shroud 16. The bottom wall 30 cooperates with the sidewall 28 to define an interior 52. The sidewall 28 can define an opening 54 that accommodates the display 32. A PCB 56 can be disposed at least partially in the interior 52. The PCB 56 can electrically support the display 32 as well as a VPD sensing module 58. The PCB 56 can be electrically connected to the PCB 40 via an electrical connector 60. The PCB 56 can be sandwiched between the light ring 26 and the bottom wall 30. The bottom wall 30 can be coupled to the light ring 26 with screws (not shown) that extend through the bottom wall 30 and are threaded into standoffs 62 that extend from the light ring 26. The attachment of the bottom wall 30 to the light ring 26 can facilitate coupling of the upper housing 12 and the lower housing 14 together.

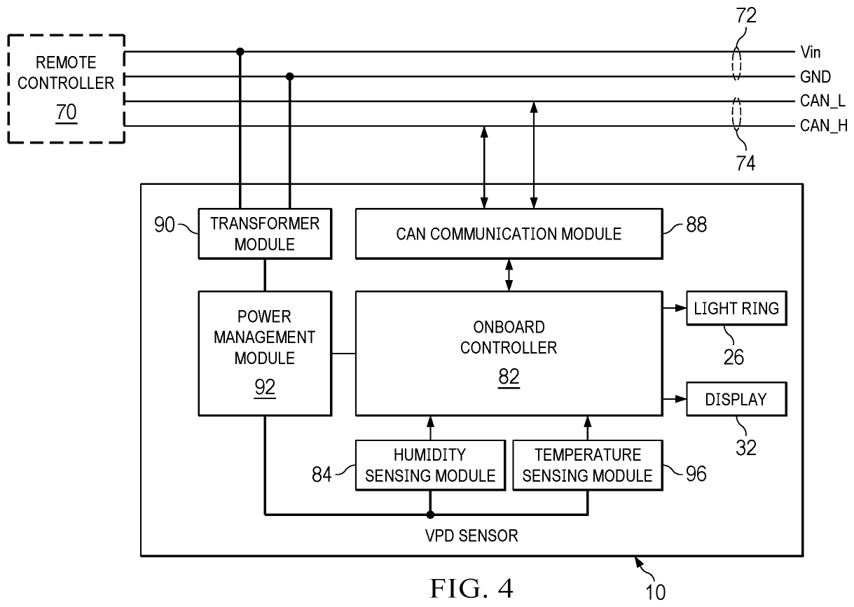

Referring now to FIG. 4, a schematic view of the VPD sensor 10 and a remote controller 70 is illustrated and will now be described. The VPD sensor 10 can be powered by a power bus 72 that is electrically coupled with the remote controller 70 and receives power therefrom. The power bus 72 can be powered by input power, typically 120 VAC, that is used to power the remote controller 70. In one embodiment, the remote controller 70 can include an internal transformer (not shown) that converts the input power received by the remote controller 70 into rated power, typically DC power, for powering the VPD sensor 10. It is to be appreciated that the VPD sensor 10 can additionally or alternatively be powered by an external power source that is routed directly to the VPD sensor 10 and thus bypasses the remote controller 70. The VPD sensor 10 can be communicatively coupled with the remote controller 70 via a controller area network (CAN) communication bus 74 such that the remote controller 70 can communicate with the VPD sensor 10 via a CAN protocol, as will be described in further detail below.

A cable (not shown) can be provided that houses both the power bus 72 and the CAN bus 74 and is plugged into each of the VPD sensor 10 and the remote controller 70. The electrical connector 22 (FIG. 1) can enable coupling of the cable to the VPD sensor 10. The electrical connector 22 can be any of a variety of connection types, such as, for example, a CNT-13 type connector, a Wieland-type connector, an RJ-45 connector, a push-pull connector, or a quick-lock connector. It is to be appreciated that the remote controller 70 can also include an electrical connector (not shown) that allows for electrical and communicative coupling of the remote controller 70 to the cable. It is to be appreciated that although only one VPD sensor 10 is shown to be connected to the remote controller 70, the remote controller 70 can support a plurality of VPD sensors that are connected together via the cable (i.e., daisy chained together) and are powered by, and communicate with, the remote controller 70 in a similar manner as described herein for the VPD sensor 10. One example of the remote controller 70 is illustrated and described in PCT Pub. No. WO2022/266475 which is hereby incorporated by reference herein in its entirety.

Still referring to FIG. 4, the VPD sensing module 58 can be configured to facilitate monitoring of the vapor pressure deficit as a function of the temperature and humidity in the surrounding air, as will be described in further detail below. An onboard controller 82 can be in signal communication with the VPD sensing module 58 and can be configured to process sensor data received therefrom as described in more detail below. The onboard controller 82 may be embodied as any type of processor capable of performing the functions described herein. For example, the onboard controller 82 may be embodied as a single or multi-core processor, a digital signal processor, a microcontroller, a general purpose central processing unit (CPU), a reduced instruction set computer (RISC) processor, a processor having a pipeline, a complex instruction set computer (CISC) processor, an application specific integrated circuit (ASIC), a programmable logic device (PLD), a field programmable gate array (FPGA), or other processor or processing/controlling circuit or controller.

The VPD sensing module 58 can include a humidity sensing module 84 and a temperature sensing module 86 that are each in signal communication with the onboard controller 82 such that the onboard controller 82 is effectively in signal communication with the VPD sensing module 58 as a whole by way of being in signal communication with each of the humidity sensing module 84 and the temperature sensing module 86. The humidity sensing module 84 can be configured to measure the humidity in the surrounding air and provide the humidity measurement as humidity data (e.g., via a transmitted analog or digital signal). The humidity sensing module 84 can measure the humidity in the surrounding air using any of a variety of techniques that may be currently known or hereafter developed. The temperature sensing module 86 can be configured to measure the temperature in the surrounding air and provide the temperature measurement as temperature data (e.g., via a transmitted analog or digital signal). The temperature sensing module 86 can measure the temperature in the surrounding air using any of a variety of techniques that may be currently known or hereafter developed.

The onboard controller 82 can then use the humidity data and the temperature data from the humidity sensing module 84 and the temperature sensing module 86 to calculate a VPD value of the surrounding air. In one embodiment, the onboard controller 82 can utilize a lookup table that maps different humidity and temperature values to specific VPD values. In some instances, the onboard controller 82 can take average the values of different humidity and temperature measurements that are received during a discrete time period to account for any erroneous measurements that might be present in some of the individual humidity and temperature measurements during that discrete time period.

The onboard controller 82 can be in signal communication with the light ring 26. When the VPD value is within a desired range, the onboard controller 82 can cause the light ring 26 to be illuminated with a colored light, such as a green light, that indicates to a user that the VPD value of the surrounding air is acceptable. When the VPD value is outside of a desired range, the onboard controller 82 can cause the light ring 26 to be illuminated with a different colored light, such as a red light, that indicates to a user that the VPD value is not acceptable. In one embodiment, the light ring 26 can include a plurality of LEDs (not shown) that are distributed throughout the light ring 26 which causes the light ring 26 to effectively glow with the particular color prescribed by the onboard controller 82. It is to be appreciated that the humidity measurement and/or the temperature measurement can additionally or alternatively cause the light ring 26 to be illuminated in different colors as a function of whether those measurements are within a desired range. It is also to be appreciated that although a light ring is described, any of a variety of suitable alternative indicating arrangements are contemplated, such as, for example, a single LED.

The onboard controller 82 can also be in signal communication with the display 32. The onboard controller 82 can cause one or more of the humidity level, the temperature, or the VPD value to be displayed on the display 32 for presentation to the user. In one embodiment, the humidity level, the temperature, and/or the VPD value displayed on the display 32 can be updated in real time. The display 32 can be an LCD screen, a segmented display or any other display type that is capable of presenting a numerical representation of the humidity level, the temperature, and/or the VPD value to a user.

The onboard controller 82 can be in signal communication with a CAN communication module 88 that is communicatively coupled with the CAN bus 74 and is configured to communicate with the remote controller 70 using a CAN architecture. The CAN architecture can facilitate bidirectional communication between the VPD sensor 10 and the remote controller 70 and between the VPD sensors themselves. The remote controller 70 can poll the VPD sensor 10 for humidity data, temperature data, and/or VPD data that might include the humidity level, temperature and/or VPD value and an indication of whether one or more of those values are outside of a predefined range. The onboard controller 82 can respond accordingly with a response message that is transmitted to the remote controller 70 and includes the specific data requested by the remote controller 70. In response, the remote controller 70 can cause the humidity level, the temperature, and/or the VPD value to be displayed on a native screen (not shown) and/or can generate an alarm for a user if the any of those values are outside of their predefined range.

The CAN architecture can also allow the remote controller 70 to detect a problem with the health of the VPD sensor 10. If the remote controller 70 detects a problem with the health of one or more of the VPD sensors 10 (e.g., based on the response message from the VPD sensor 10), such as a communication problem or as a result of a fault code transmitted to the CAN bus 74 by the remote controller 70, the remote controller 70 can notify a user of the problematic VPD sensor by activating an indicator (e.g., a light or an audible sound) on the problematic VPD sensor (e.g., one of LEDs 24), activating an indicator on a surrounding VPD sensor (e.g., intermittently illuminating an indicator on an adjacent VPD sensor to the problematic VPD sensor (e.g., an immediately upstream or downstream sensor)), and/or displaying the unique ID of the problematic VPD sensor on the native display.

Still referring to FIG. 4, the VPD sensor 10 can include a transformer module 90 that is electrically connected on one side to the power bus 72 and at the other side to each of the VPD sensing module 58 and the onboard controller 82 to facilitate powering thereof from the power bus 72. The transformer module 90 can be configured to transform the power from the power bus 72 into usable power for powering the VPD sensing module 58 and the onboard controller 82. In one embodiment, the transformer module 90 can be configured to generate different DC voltages (e.g., 5 VDC, 12 VDC, 15 VDC) for powering the VPD sensing module 58 and the onboard controller 82. In one embodiment, the transformer module 90 can comprise an isolated DC/DC converter. A power management module 92 can be provided downstream of the transformer module 90 and can be configured to condition the power signal delivered from the transformer module 90 before it reaches the VPD sensing module 58 and the onboard controller 82.

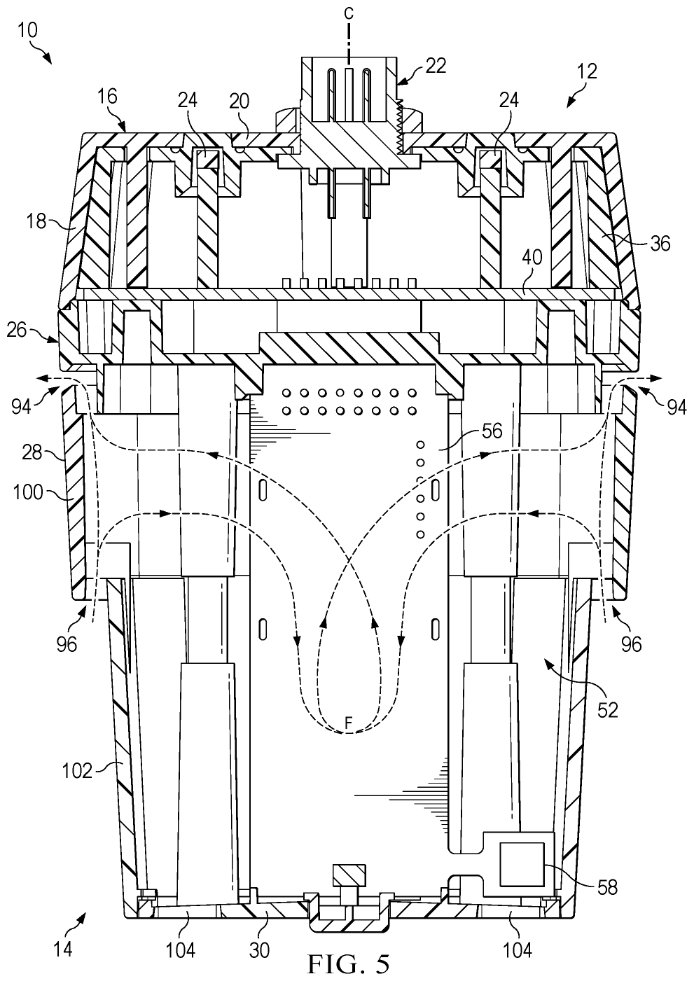

Referring now to FIG. 5, the upper housing 12 and the lower housing 14 can be spaced from each other along an outer perimeter P1 (FIGS. 1 and 2) and relative to a centerline C such that the upper and lower housings 12, 14 cooperate to define an air gap 94 therebetween that is in fluid communication with the interior 52 of the lower housing 14. In particular, the air gap 94 can be defined by a lowermost portion of the upper housing 12 at the perimeter P1 and an uppermost portion of the lower housing 14 at the perimeter P1. In one embodiment, as illustrated in FIG. 5, the lowermost portion of the upper housing 12 is shown to be defined by the light ring 26 and the uppermost portion of the lower housing 14 is shown to be defined by the sidewall 28 such that the air gap 94 is defined by the light ring 26 and the sidewall 28. In such an embodiment, the sidewall 28 can be laterally spaced from the light ring 26 (e.g., in a direction that is perpendicular to the centerline C) such that the air gap 94 is able to extend downwardly from the upper housing 12 and into the interior 52. It is to be appreciated, however, that the definition of the air gap 94 should not be limited to what is shown in FIG. 5. For example, in embodiments where the upper housing 12 might be devoid of a light ring (e.g., 26), another part of the upper housing 12, such as the sidewall 18, could cooperate with the lower housing 14 to define an air gap. In addition, although the air gap 94 is shown to extend entirely circumferentially around the VPD sensor 10, the air gap 94 might only extend partially circumferentially around the VPD sensor 10.

The sidewall 28 of the lower housing 14 can define a pair of openings 96 that are disposed beneath the air gap 94 (e.g., when viewed relative to the centerline C) and that are in fluid communication with the interior 52. Each opening 96 can cooperate with the air gap 94 to allow for the surrounding air to flow through the interior 52 along a fluid pathway F that extends into the interior 52 and between the openings 96 and the air gap 94. The openings 96 can be disposed on opposite sides of the sidewall 28. In one embodiment, each of the openings 96 can extend circumferentially along part of the sidewall 28.

The sidewall 28 of the lower housing 14 can include an upper portion 100 that is disposed adjacent to the upper housing 12 and a lower portion 102 that is disposed adjacent to the bottom wall 30. The upper and lower portions 100, 102 can be spaced from each other and can thus cooperate to define the openings 96 such that the fluid pathway F at the openings 96 is routed between the upper and lower portions 100, 102. The PCB 56 can be positioned with respect to the upper and lower portions 100, 102 such that the VPD sensing module 58 is disposed in the interior 52 adjacent to the lower portion 102.

Because the lower portion 102 is disposed beneath the openings 96, when the surrounding air is introduced through the openings 96 and flows over the lower portion 102, the surrounding air can be at least at least partially diverted into the lower portion 102 before it is exhausted through the air gap 94. This diversion into the lower portion 102 can cause the surrounding air to take a tortuous path through the lower portion 102 which effectively mixes the surrounding air within the interior 52. The mixed air can enhances the cooling of the electrical components air and can mitigate the occurrence of humidity and temperature “hot spots” at the VPD sensing module 58 that might cause erroneous humidity and temperature detections. In addition, by locating the VPD sensing module 58 towards the bottom of the lower portion 102, the mixed air that reaches the VPD module 58 there is more likely to be more thoroughly mixed which can further enhance the accuracy of the VPD sensing module 58.

Still referring to FIG. 5, the upper portion 100 is shown to be laterally offset relative to the lower portion 102 (e.g., in a direction that is perpendicular to the centerline C) such that a perimeter P2 of the upper portion 100 at the openings 96 (see FIGS. 1 and 2) is greater than a perimeter P3 of the lower portion 102 at the openings 96. The upper portion 100 can therefore effectively serve as a hood that overhangs the lower portion 102 such that the openings 96 face towards the bottom wall 30. When surrounding air is introduced from underneath the VPD sensor 10, the surrounding air is encouraged to flow into the openings 96 and through the interior 52 rather than around the VPD sensor 10 (e.g., via a chimney effect. For example, as the surrounding air rises around the VPD sensor 10 (e.g., due to convention current), the upper portion 100 can encourage the rising air into the openings 96 as it passes along the lower portion 102. Additionally, during operation of the VPD sensor 10, the heat generated by the internal electrical components can cause the interior 52 to be hotter than the surrounding air which can further encourage the surrounding air to be drawn through the openings 96 and into the interior 52.

Referring now to FIGS. 2, 3 and 5, the bottom wall 30 can include a pair of vents 104 that are in fluid communication with the interior 52. The vents 104 can allow for further introduction of the surrounding air into the interior 52 to enhance the cooling effects of the surrounding air relative to the internal electrical components of the VPD sensor 10 and/or to enhance the mixing of the surrounding air that is introduced to the interior 52. Additional views of the VPD sensor 10 are shown in FIGS. A-H provided as an Appendix.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein but can be employed in any number of applications and equivalent devices by those of ordinary skill in the art. Rather, it is hereby intended that the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is claimed is:

1. A vapor pressure deficit sensor for a horticultural environment, the vapor pressure deficit sensor comprising:
an upper housing;
a lower housing coupled with the upper housing and comprising a lower sidewall and a bottom wall that cooperate to define an interior; and
a vapor pressure deficit sensing module coupled with the lower housing and disposed in the interior, wherein:
the upper housing and the lower housing are spaced from each other along a centerline to define an air gap therebetween that is in fluid communication with the interior; and
the lower sidewall defines an opening that is in fluid communication with the interior and cooperates with the interior and the air gap to define a fluid pathway that extends through the interior and between the opening and the air gap.

2. The vapor pressure deficit sensor of claim 1 wherein the lower sidewall comprises:
an upper portion adjacent to the upper housing; and
a lower portion adjacent to the bottom wall, wherein the upper portion is laterally offset from the lower portion relative to the centerline; and
the opening is defined by the upper portion and the lower portion of the sidewall.

3. The vapor pressure deficit sensor of claim 2 wherein the opening extends circumferentially along at least part of the lower sidewall.

4. The vapor pressure deficit sensor of claim 1 wherein the air gap extends circumferentially around the upper housing and the lower housing.

5. The vapor pressure deficit sensor of claim 4 wherein the air gap extends entirely circumferentially around the upper housing and the lower housing.

6. The vapor pressure deficit sensor of claim 1 wherein the upper housing further comprises:
an upper sidewall; and
a light ring attached to the upper sidewall adjacent to the lower housing such that the light ring at least partially defines the air gap.

7. The vapor pressure deficit sensor of claim 6 wherein the light ring is routed entirely circumferentially around the upper sidewall.

8. The vapor pressure deficit sensor of claim 1 wherein the bottom wall defines a vent that is in fluid communication with the interior of the lower housing.

9. A vapor pressure deficit sensor for a horticultural environment, the vapor pressure deficit sensor comprising:
a humidity sensing module configured to measure the humidity of surrounding air;
a temperature sensing module configured to measure the temperature of surrounding air;
an onboard controller in signal communication with the humidity sensing module and the temperature sensing module and configured to determine a vapor pressure deficit value based upon the measured humidity and the measured temperature; and
a CAN communication module in signal communication with the onboard controller and configured to facilitate bidirectional communication with a remote controller via a controller area network architecture, wherein the onboard controller is configured to transmit one or more of the humidity measurement, the temperature measurement, or the vapor pressure deficit value to the remote controller via the CAN communication module.

10. The vapor pressure deficit sensor of claim 9 further comprising a display screen configured to display information about the environmental parameter.

11. A vapor pressure deficit sensor for a horticultural environment, the vapor pressure deficit sensor comprising:
an upper housing;
a lower housing coupled with the upper housing and comprising a lower sidewall and a bottom wall that cooperate to define an interior; and
a vapor pressure deficit sensing module coupled with the lower housing and disposed in the interior, the vapor pressure deficit sensing module comprising:
a humidity sensing module configured to measure the humidity of surrounding air;
a temperature sensing module configured to measure the temperature of surrounding air;
an onboard controller in signal communication with the humidity sensing module and the temperature sensing module and configured to determine a vapor pressure deficit value based upon the measured humidity and the measured temperature; and
a CAN communication module in signal communication with the onboard controller and configured to facilitate bidirectional communication with a remote controller via a controller area network architecture, wherein the onboard controller is configured to transmit one or more of the humidity measurement, the temperature measurement, or the vapor pressure deficit value to the remote controller via the CAN communication module, wherein:
the upper housing and the lower housing are spaced from each other along a centerline to define an air gap therebetween that is in fluid communication with the interior; and
the lower sidewall defines an opening that is in fluid communication with the interior and cooperates with the interior and the air gap to define a fluid pathway that extends through the interior and between the opening and the air gap.

12. The vapor pressure deficit sensor of claim 11 wherein the lower sidewall comprises:
an upper portion adjacent to the upper housing; and
a lower portion adjacent to the bottom wall, wherein the upper portion is laterally offset from the lower portion relative to the centerline; and
the opening is defined by the upper portion and the lower portion of the sidewall.

13. The vapor pressure deficit sensor of claim 12 wherein the opening extends circumferentially along at least part of the lower sidewall.

14. The vapor pressure deficit sensor of claim 11 wherein the air gap extends circumferentially around the upper housing and the lower housing.

15. The vapor pressure deficit sensor of claim 14 wherein the air gap extends entirely circumferentially around the upper housing and the lower housing.

16. The vapor pressure deficit sensor of claim 11 wherein the upper housing further comprises:
an upper sidewall; and
a light ring attached to the upper sidewall adjacent to the lower housing such that the light ring at least partially defines the air gap.

17. The vapor pressure deficit sensor of claim 16 wherein the light ring is routed entirely circumferentially around the upper sidewall.

18. The vapor pressure deficit sensor of claim 11 wherein the bottom wall defines a vent that is in fluid communication with the interior of the lower housing.
